(19) United States
(12) Patent Application Publication
(10) Pub. No.: US 2026/0210961 A1
(43) Pub. Date: Jul. 23, 2026

(54) SYSTEMS AND METHODS FOR IDENTIFYING GPCR MODULATORS AND OTHER AGENTS

(71) Applicants: President and Fellows of Harvard College, Cambridge, MA (US); Cambridge Enterprise Limited, Cambridgeshire, Cambridge (GB)

(72) Inventors: David A. Weitz, Cambridge, MA (US); Karla Milcic, Cambridge, MA (US); Tuomas Pertti Jonathan Knowles, Cambridge (GB); Anqi Chen, Cambridge, MA (US)

(73) Assignees: President and Fellows of Harvard College, Cambridge, MA (US); Cambridge Enterprise Limited, Cambridgeshire, Cambridge (GB)

(21) Appl. No.: 19/139,319

(22) PCT Filed: Dec. 14, 2023
(86) PCT No.: PCT/US2023/084135

§ 371 Date: Jun. 13, 2025

Related U.S. Application Data
(60) Provisional application No. 63/432,600, filed on Dec. 14, 2022.
(60) Provisional application No. 63/432,606, filed on Dec. 14, 2022.

(51) Int. Cl. G01N 33/566; G01N 33/68

(52) U.S. Cl. CPC G01N 33/566; G01N 33/68; G01N 2333/726; G01N 2474/00

(57) ABSTRACT

System and methods for identifying agents (e.g., proteins, peptides) that modulate G-protein coupled receptors (GPCRs) are generally described. For some embodiments, the agent is a nanobody that modulates the GPCR and may either act as an agonist (e.g., activate) or antagonist (e.g., deactivate) to the GPCR.

### RELATED APPLICATIONS

This application claims the benefit of U.S. Provisional Patent Application Ser. No. 63/432,606, filed Dec. 14, 2022, and of U.S. Provisional Patent Application Ser. No. 63/432,600, filed Dec. 14, 2022. Each of these is incorporated herein by reference in its entirety.

### TECHNICAL FIELD

System and methods for identifying agents (e.g., proteins) that modulate G protein-coupled receptors (GPCRs) are generally described.

### BACKGROUND

G protein-coupled receptors (GPCRs) belong to a large family of signaling proteins that mediate cellular responses to many hormones, metabolites, cytokines, and neurotransmitters. More than 800 genes comprise this receptor family, which modulate several signaling processes involved in behavior, blood pressure regulation, cognition, immune response, mood, smell, and taste.

GPCRs are categorized into six classes based on sequence and function, namely Class A—rhodopsin-like receptors, Class B—secretin family, Class C—metabotropic glutamate receptors, Class D—fungal mating pheromone receptors, Class E—cAMP receptors, and Class F—frizzled (FZD) and smoothened (SMO) receptors. GPCR members share a common seven transmembrane (7TM) architecture linked by three extracellular (ECL) and three intracellular (ICL) loops. However, they have low sequence identity and possess different extracellular N-terminal domains and diverse ligand-binding pockets. The major disease indications for GPCR modulators show a shift towards diabetes, obesity, and Alzheimer's disease, while other central nervous system disorders remain highly represented. There are also some non-olfactory GPCRs that are yet to be explored in clinical trials and have broad untapped therapeutic potential, particularly in genetic and immune system disorders. Accordingly, improved systems and methods are desired.

### SUMMARY

System and methods for identifying agents (e.g., proteins, peptides, antibodies) that modulate G-coupled protein receptors (GPCRs) are generally described. For some embodiments, the agent is a nanobody that modulates the GPCR and may either act as an agonist (e.g., activate) or antagonist (e.g., deactivate) to the GPCR. The subject matter of the present disclosure involves, in some cases, interrelated products, alternative solutions to a particular problem, and/or a plurality of different uses of one or more systems and/or articles.

In one aspect, a method for determining proteins that bind to GPCRs is described, the method comprising containing one or more proteins within a microfluidic droplet, wherein at least one of the proteins is suspected of being capable of binding to a signaling entity; binding at least one of the proteins contained within the microfluidic droplet to a GPCR receptor coupled to the signaling entity, wherein the binding activates the signaling entity; and determining a signal from the signaling entity. In some such aspects, binding activates the signaling entity and/or initiates a GPCR cascade that activates the signaling entity.

In another aspect, a method for determining proteins that bind to GPCRs is described, the method comprising containing one or more proteins within a microfluidic droplet, wherein at least one of the proteins is suspected of being capable of binding to a signaling entity; binding at least one of the proteins contained within the microfluidic droplet to a GPCR receptor coupled to the signaling entity; and determining a signal from the signaling entity. In some such aspects, binding deactivates the signaling entity and/or initiates a GPC cascade that deactivates the signaling entity.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 presents a schematic of a method for selecting genetic material that encodes for nanobodies that bind to and/or modulate the activity of GPCRs from a gene library, according to some embodiments;

FIGS. 2A-2C illustrates various embodiments of methods used for screening proteins and/or the genetic material that encode for proteins that bind and/or modulate the activity of GPCRs;

FIG. 2D is a schematic illustration of a protein nanobody suspected of binding and modulating the activity of a GPCR, according to some embodiments;

FIG. 2E is a schematic illustration of a complex comprising a protein nanobody suspected of binding and/or modulating the activity of a GPCR, according to some embodiments;

FIG. 2F is a schematic illustration of a cell configured to report the activity of a GPCR protein that has been encapsulated within a semipermeable membrane, along with a nanobody suspected of binding to the cell, according to some embodiments;

FIG. 3 is a schematic diagram illustrating one example of a prescreening process in which a nanobody is bound to the mRNA that encodes for the nanobody, and a cDNA of the mRNA that encodes mRNA/nanobody is synthesized, according to some embodiments;

FIG. 4A shows a schematic and a microscope image of a live cell assay in which the cell reports the activity of a nanobody of interest and the cell are grown within a semipermeable microcapsule, according to one set of embodiments; and

FIG. 4B shows microscope images and a plot demonstrating reporter function of the cells encapsulated in a semipermeable membrane, according to one set of embodiments.

### DETAILED DESCRIPTION

Systems and methods for identifying agents (e.g., proteins, peptides, nanobodies) that can bind and/or modulate the activity of G protein-coupled receptors (GPCRs) are generally described below. Because GPCRs regulate numerous cellular responses that impact a variety of bodily functions, molecules that target and bind to GPCRs may mediate cellular responses, which may have important implications regarding diseases. That is, molecules that target and bind to GPCRs can offer new approaches to regulating cellular responses, which makes such molecules promising candidates for therapeutics. For example, a molecule that may selectively bind to a GPCR that regulates a hormone in the body may offer a new treatment for diseases for hormonal imbalances related to the hormone. However, identifying molecules that target and bind specific GPCRs has been challenging due to the large number and variety of structures of GPCRs. In addition, since GPCRs are transmembrane proteins, it has been challenging to measure the in situ activity of these GPCRs because membrane proteins often have low stability outside of their native membrane environment, making them difficult to identify with conventional systems and methods.

In view of the foregoing, described herein are various systems and methods for identifying molecules (e.g., proteins, peptides, antibodies, nanobodies) that bind with GPCRs that may modulate or regulate the cell signaling pathways that are mediated by GPCRs. This may be useful, in some embodiments, for developing treatments for diseases (e.g., by using the molecules that bind to GPCRs to regulate cell signaling pathways mediated by GPCRs). As described by certain embodiments of this disclosure, agents, such as proteins, can be identified that both bind and modulate (e.g., activate, deactivate) the activity of GPCRs. Advantageously, some of the systems and methods may identify activity of GPCRs while within a cell membrane (or some other native configuration of the GPCR), for example, such that a determination of the GPCR activity can better be determined relative to existing GPCR assays, which are not able to correlate both GPCR binding and activity. In some cases, the systems and methods described below and elsewhere herein can be used to correlate both binding of an agent to the GPCR and the activity of the GPCR after binding of the agent. In this manner, in certain embodiments, binding of a particular agent (e.g., a nanobody that targets the GPCR) may also advantageously determine if binding modulates the GPCR to produce a desired functionality, rather than merely binding, without producing the desired functionality of the GPCR, as is the case for certain existing systems and methods used to screen GPCRs.

Accordingly, some embodiments are directed to systems and methods for detecting proteins (e.g., nanobodies) that bind to transmembrane proteins (e.g., GPCRs). In some embodiments, a gene library may be translated and transcribed into a plurality of proteins. Individual proteins from the plurality of proteins may be bound (e.g., covalently bound, non-covalently bound) to the nucleic acid from which they were encoded (e.g., translated) to form one or more proteins to which the nucleic acid encoding for that protein is bound to the protein. In some embodiments, the protein-nucleic acid complexes can be exposed to GPCRs of interest, wherein the complex binds to the GPCRs. Advantageously, the complexes which modulate the GPCRs in a desired manner (e.g., generating a stronger signal relative to other proteins, activating one or more cascades associates with the GPCR) may be selected, thereby providing a selective advantage for proteins that bind to the GPCR(s) of interest and its corresponding nucleic acid which encodes for the protein of interest (e.g., mRNA ligated to the nanobody). In some cases, the nucleic acid (e.g., a first nucleic acid) that encodes for the protein and/or a complement of this nucleic acid (e.g., a second nucleic acid, cDNA of the first nucleic acid) may be amplified to generate genetic material from a protein of interest, at least in part based on binding the protein of interest with nucleic acid that encodes for that protein of interest. In some such embodiments, proteins of interest (attached to nucleic acids coding for the proteins of interest) can be collected into a library (e.g., a first library) and the library (e.g., proteins and/or nucleic acids/genes from library) can be translated and/or transcribed again to produce a secondary library, where the second library is smaller than the first library. In some such embodiments, this may be repeated (e.g., a second library reduced to a third library, a third library reduced to a fourth library, etc.), wherein the number of genes in each library is successively reduced.

While various embodiments described herein are in the context of a protein binding to a GPCR, it will be understood in view of the following disclosure that the system and methods are suitable for screening a variety of agents against a variety of binders. For example, in some embodiments, the proteins are screened against a transmembrane protein that does not comprise a GPCR. Examples of non-GPCR screenings are described elsewhere herein. Furthermore, it will be understood that the agent need not comprise a protein, and other binding agents are possible, as described elsewhere herein.

In some embodiments, a protein library and/or a gene library may be screened using a droplet microfluidic platform. By encapsulating the protein and/or genes (e.g., a first nucleic acid, a second nucleic acid) into individual droplets comprising in vitro translation and/or transcription reagents with cells and a substrate of interest (e.g., GPCRs, other receptors), the genes may be translated and transcribed into proteins (e.g., nanobodies) within each droplet. Therefore, in some embodiments, proteins that were transcribed from the gene library may be exposed to substrates of interest, such as GPCRs within droplets. In some cases, the droplets comprise one or more cells expressing the GPCR on the surface of the cell. However, it will be understood that non-spherical shapes and/or non-aqueous based droplets are contemplated (e.g., rods, capsules, microgels, microcapsules).

In some embodiments, it may be advantageous to modify a substrate of interest (e.g., a transmembrane protein, a GPCR) with one or more signaling entities (e.g., a molecule or moiety configured for Forster resonance energy transfer, FRET). In this manner, the activity of the substrate of interest may be coupled to binding of the substrate. In some cases, upon binding between a protein and the substrate of interest within a droplet, the signaling entity may produce a signal that indicates the droplet contains proteins that bind to and modulate (e.g., activate, deactivate) the substrate of interest. The signal may be a light or electromagnetic radiation-based signal, such as fluorescence, phosphorescence, or some other chemiluminescence process. In accordance with some embodiments, droplets containing proteins that bind and modulate substrates of interest can be sorted and/or combined (e.g., droplet sorting, droplet merging) to provide proteins that target substrates of interest and/or corresponding nucleic acids that encode for the proteins. In some cases. these proteins may be useful for regulating cellular responses (e.g., inhibiting a cellular response, treating a disease associated with the cellular response).

Different substrates of interest may be screened, in accordance with some embodiments. In some cases, the substrate of interest comprises a transmembrane protein (e.g., a GPCR). A variety of transmembrane proteins may be screened and is not limited by the class of the transmembrane protein. In some embodiments, the transmembrane protein comprises a 7-TM transmembrane protein. In certain embodiments, the transmembrane protein comprises a GPCR. It may be advantageous to use a GPCR of interest in some cases, wherein the GPCR mediates a cellular response that is related to a disease. In some cases, the cellular response mediated by the GPCR is suspected of being involved with a disease. In some embodiments, the cellular response mediated by the GPCR is known to be directly involved with a disease. In certain embodiments, the cellular response mediated by a GPCR is known to cause a disease. In some embodiments, the GPCRs are expressed in a plurality of cells. However, in other embodiments, GPCRs are expressed in a cell-free medium. In some cases, the GPCRs comprise human-derived GPCRs. In some embodiments, human-derived GPCRs are expressed in a plurality of mammalian cells. In some embodiments, a plurality of substrates of interest may be screened (e.g., for a multiplexed screening). In some such embodiments, the plurality of substrates of interest may include one or more GPCR targets and/or one or more other substrates (e.g., other transmembrane proteins) to be screened.

Various drugs are known that target GPCR receptors, some of which are described elsewhere herein. In some embodiments, the GPRC comprises targets OPRD, OPRM 5HT2A, 5HT1A, DRD2, 5HT7R GLP1R NK1R NK1R PI2R SMO PTHR1 5HT2A P2Y12 PAR1, PAR3, PAR4 ADRB2 PTH1R GLP1R OX2R, OX1R MTR1A, MTR1B OPRM, OPRD OPRM OPRM ADRB1-3, ADA2A/B/C, ADA1A/B/D CASR 5HT1A, 5HT2A, DRD2 DRD3, DRD2 5HT2A, and/or 5HT1A, and, in some embodiments these targets can be screened.

While many of the embodiments described herein involve modulating a GPCR protein, it should be understood that other substrates of interest may also be identified, screened, and/or have their activities modulated.

As mentioned above, in some embodiments, a method for identifying or detecting agents that selectively bind to and/or modulate the activity of transmembrane proteins (e.g., GPCRs) of interest has been developed. For various embodiments, the agent is a protein (e.g., a nanobody) that binds and/or modulates the activity of the transmembrane protein. However, for other embodiments, other binding agents are screened. A variety of chemical and/or biological species may act as an agent that binds to the transmembrane protein such as ligands (e.g., small molecules, antibodies, antigens). For various embodiments, the agent comprises a protein (e.g., a protein of interest). In some such embodiments, the protein comprises a nanobody. Advantageously, nanobodies are relatively small (e.g., smaller than antibodies) and comprise a single peptide chain, and hence does not require pairing of chains (e.g., during translation, during screening, during pre-screening). Nanobodies may also be less susceptible to certain issues associated with antibodies, such as solubility and/or aggregation issues, and may bind more strongly to active sites of proteins (e.g., a cavity of a protein) compared to antibodies. Without wishing to be bound by any theory, nanobodies can often infiltrate solid tumors, provide a high degree of thermal stable, and, in some cases, can also survive exposure to gastric fluids. However, as noted above, it will be understood that other agents may be screened and/or identified, such as other proteins or other species (e.g., antigen

In some embodiments, one of more proteins may selectively bind with transmembrane proteins of interest and/or other species not of interest. Selective binding between one or more proteins and the transmembrane protein is described elsewhere herein. In some embodiments, a nucleic acid that encodes for the nanobodies comprises GPCR promoters and/or terminators. In some cases, one or more nanobodies bind to and/or modulate the activity of a GPCR of interest.

In some embodiments, systems and methods for determining proteins (e.g., nanobodies) that bind and/or modulate (e.g., activate, deactivate) a transmembrane protein (e.g., a GPCR) include a gene library (e.g., a nucleic acid library, a DNA library). In some such embodiments, the library is suspected of containing at least one sequence that encodes for a protein (e.g., a nanobody) that binds to and modulates the activity of a transmembrane protein. In some embodiments, using a gene library facilitates determining proteins that bind to transmembrane proteins. In some embodiments, using a gene library facilitates determining nanobodies that activate and/or deactivate GPCRs. In some embodiments, the gene library comprises genes encoding for variants of one or more protein. In some embodiments, the gene library comprises genes that encode for variants of one or more nanobodies.

In some cases, pre-screening a gene library comprising nucleic acids may be useful for providing nucleic acids (e.g., DNA and/or mRNA) encoding for one or more proteins (e.g., a nanobody) suspected of being capable of binding to and/or modulating a transmembrane protein (e.g., a GPCR). In accordance with some embodiments, pre-screening a gene library comprises selecting a plurality of nucleic acids from the gene library, wherein a number of nucleic acids in the plurality of nucleic acids is less than a second number of nucleic acids that initially comprise the gene library. In some embodiments, a first library is screened. In some embodiments, a second library is screened. In some such embodiments, the second library comprises less genes (e.g., less nucleic acids) than the first nucleic acid.

The gene library may, at least initially, comprise any number of genes encoding for proteins (and/or variants of proteins). In some cases, the number of genes within the library is relatively large, being greater than or equal to 10⁵, greater than or equal to 10⁶, greater than or equal to 10⁷, greater than or equal to 10⁸, greater than or equal to 10⁹, greater than or equal to 10¹⁰, greater than or equal to 10¹¹, greater than or equal to 10¹², greater than or equal to 10¹³, greater than or equal to 10¹⁴, greater than or equal to 10¹⁵. genes. In some cases, the library comprises less than or equal to 10¹⁵, less than or equal to 10¹⁴, less than or equal to 10¹³, less than or equal to 10¹², less than or equal to 10¹¹, less than or equal to 10¹⁰, less than or equal to 10⁹, less than or equal to 10⁸, less than or equal to 10⁷, less than or equal to 10⁶, or less than or equal to 10⁵ genes. Combinations of the foregoing ranges are possible (e.g., a gene library comprising greater than or equal to 10⁸ and less than or equal to 10¹⁵ genes). Other ranges are also possible, as this disclosure is not so limited.

In some cases, the gene library may comprise DNA. In some such embodiments, the DNA is transcribed to make RNA (e.g., mRNA). In some cases, transcribing the DNA involves using T7 promoters, ribosomal binding sites, and/or T7 terminators. Similarly, when the gene library comprises RNA, the RNA may be reverse transcribed into cDNA of the RNA. In some such embodiments, the cDNA base pairs with the RNA.

Screening a gene library to select nucleic acids (e.g., DNA, RNA) which encode for one or more proteins (e.g., nanobodies) suspected of being capable of binding a transmembrane protein (e.g., a GPCR) can be useful, and screening the gene library can be done in a variety of suitable approaches. For example, in some embodiments, the gene library comprising a first nucleic acid (e.g., mRNA) suspected of encoding for a protein (e.g., a nanobody) which binds a transmembrane protein (e.g., a GPCR) may be used. In some embodiments, the first nucleic acid may comprise mRNA. In some embodiments, the mRNA is suspected of encoding for a protein configured to bind to a GPCR. In some embodiments, the mRNA is suspected of encoding for a nanobody configured to bind to the GPCR. In some cases, the mRNA comprises a 7-TM promoter and/or terminator.

In some cases, a first nucleic acid comprising mRNA is bound to a linking reagent. In some cases, wherein mRNA is translated to synthesize a protein (i.e., a nanobody) for which it encodes, the linking reagent may enable the cleavage of the translated protein from the mRNA and/or transfer the protein onto the linking reagent. In some embodiments, the linking reagent comprises puromycin and/or a DNA linker. In some cases, the linking reagent comprises tRNA. The use of other linking reagents is also possible.

In some embodiments, a protein (e.g., a nanobody) may be produced from a first nucleic acids (e.g., mRNA). In some cases, the first nucleic acid may be translated to form a protein of interest (e.g., a nanobody suspected of binding and/or modulating a GPRC). In accordance with some embodiments, following translation of the first nucleic acid to form the protein of interest, the protein of interest is attached to the first nucleic acid via the linking reagent. In this manner, in some embodiments, at least one of the proteins forms a complex comprising at least the first nucleic acid, the linking reagent, and the at least one protein, wherein the at least first nucleic acid encodes for the at least one protein. In some embodiments, mRNA is translated to produce a nanobody, wherein puromycin that is ligated to the mRNA cleaves the nanobody from the mRNA and transfers the nanobody to the puromycin, thus forming a complex comprising mRNA, the nanobody for which the mRNA encodes, and puromycin.

In some embodiments, the method further comprises synthesizing a second nucleic acid from a first nucleic acid to form a complex comprising the first nucleic acid, the second nucleic acid, and a protein of interest, wherein the first nucleic acid encodes for the protein of interest. In some embodiments, the second nucleic acid comprises DNA (e.g., DNA complimentary to the RNA from it was transcribed, cDNA). In some cases, the second nucleic acid comprises a cDNA strand of the first nucleic acid comprising mRNA. In some cases, the method comprises reverse transcribing the mRNA to obtain the cDNA strand. In some such embodiments, the cDNA strand base pairs with the mRNA. In some embodiments, the linking reagent may be transferred from the first nucleic acid to the second nucleic acid. In some embodiments, the method comprises reverse transcription of the mRNA that is ligated to the puromycin and to the protein for which it encodes to form the cDNA, wherein the puromycin is then transferred to the cDNA. In accordance with some embodiments, introducing a step of synthesizing the second nucleic acid may be useful because it can stabilize the complex comprising the first nucleic acid, the protein for which the first nucleic acid encodes, puromycin, and the second nucleic acid.

To determine which complexes (e.g., mRNA-nanobody complexes) selectively bind to transmembrane proteins (e.g., GPCRs), in some embodiments, the complexes can be exposed to cells. In some embodiments, the cells are mammalian derived. Non-limiting examples of mammalian-derived cells include cells obtained from a human, a non-human primate, a cow, a horse, a pig, a sheep, a goat, a dog, a cat, or a rodent such as a mouse, a rat, a hamster, or a guinea pig. In some cases, the cells comprise transmembrane proteins. In some cases, the cells comprise GPCRs. In some embodiments, the cells comprise GPCRs of interest, wherein GPCRs of interest modulate a GPCR signaling cascade that is involved with a disease. In some such cases, complexes that selectively bind to GPCRs may bind to the cells comprising GPCRs of interest, thereby modulating the GPCR signaling cascade.

Binding can be achieved in a variety of suitable embodiments. In some embodiments, binding comprises lowering the total free energy of the complexes and the cells (i.e., a negative ΔG value). In some cases, selective binding comprises forming a complex (e.g., a nanobody with a first GPCR) with a lower total free energy (e.g., a more negative ΔG value) than other possible complexes (e.g., a nanobody with a second GPCR, a nanobody with a third GPCR, and so forth). In some embodiments, binding comprises hydrogen bonding between the complexes and the cells comprising GPCRs. In some cases, binding comprises van der Waals forces attracting the complexes and the cells comprising GPCR. In some embodiments, binding comprises forming a bond (e.g., a covalent and/or an ionic bond) between the complexes and the cells comprising GPCRs. Other binding methods, as well as combinations of the foregoing binding methods (e.g., hydrogen bonding and forming a bond between the complexes and the cells comprising GPCRs), are possible. Upon binding of the complex to the cells comprising GPCRs, a GPCR signaling cascade may be modulated (e.g., activated, deactivated). Modulating signaling cascades are described in more detail elsewhere herein.

In some embodiments, the cells comprising GPCRs to which the complexes are bound are immobilized on a solid support. A variety of solid supports are suitable for use. In some embodiments, the solid support comprises a glass substrate. In some cases, the solid support comprises a microtiter plate. In some embodiments, the solid support comprises a polymer. Non-limiting examples of polymer substrates include PTFE, polystyrene, and polypropylene. Other solid supports are possible, as this disclosure is not so limited.

In some embodiments, upon exposing complexes (e.g., complexes comprising mRNA and nanobodies for which the mRNA encodes) to cells, binding of the complexes to the cells immobilized on a solid support may occur. Exposing complexes to cells may comprise introducing a solution (e.g., an aqueous solution, a buffered solution) comprising the complexes to the cells, wherein the cells may be immobilized on the solid support. In other cases, exposing the complexes to cells may comprise drop casting the solution comprising the complexes onto the solid support wherein the cells are immobilized and allowing the solution to dry (e.g., evaporate). Other methods for exposing the complexes to the cells immobilized on the solid support are possible, as this disclosure is not so limited.

To determine complexes that bind to cells, it may be useful to wash the cells after exposing them to the complexes. Washing the cells, in some cases, comprises flowing solution (e.g., an aqueous solution, a buffered solution, phosphate-buffered solution) over the cells immobilized on a solid support. In some embodiments, washing the cells comprises introducing and then removing at least one aliquot of solution on the solid support on which the cells are immobilized. Other methods for washing the cells are possible. In some embodiments, washing the cells after exposing the cells to the complexes removes complexes that do not bind to the cells comprising GPCRs. In some embodiments, after washing the cells, only complexes that bind to the cells comprising GPCRs remain on the cells. In some embodiments, complexes that bind to the cells comprise nanobodies that modulate (e.g., activate, deactivate) a GPCR signaling cascade that is mediated by the GPCRs. Modulating the activity of a signaling cascade is described in more detail elsewhere herein.

After washing the cells, the number of remaining complexes comprising nucleic acids (e.g., mRNA) that encode for proteins (e.g., nanobodies) that bind to transmembrane proteins (e.g., GPCRs) may be less than the number of genes initially in the gene library. In some cases, the number of complexes bound to the cells is relatively large, being greater than or equal to 1, greater than or equal to 10¹, greater than or equal to 10², greater than or equal to 10³, greater than or equal to 10⁴, greater than or equal to 10⁵, greater than or equal to 10⁶, greater than or equal to 10⁷, greater than or equal to 10⁸, greater than or equal to 10⁹, greater than or equal to 10¹⁰, greater than or equal to 10¹¹, or greater than or equal to 10¹² complexes bound to cells. In some cases, the number of complexes bound to the cells is less than or equal to 10¹², less than or equal to 10¹¹, less than or equal to 10¹⁰, less than or equal to 10⁹, less than or equal to 10⁸, less than or equal to 10⁷, less than or equal to 10⁶, less than or equal to 10⁵, less than or equal to 10⁴, less than or equal to 10³, less than or equal to 10², less than or equal to 10¹, or less than or equal to 1 complex bound to the cells. Combination of these ranges is possible (e.g., greater than or equal to 10² and less than or equal to 10⁵). Other ranges are also possible.

Following the washing step, complexes comprising nucleic acids (e.g., cDNA and/or mRNA) that encode for proteins (e.g., nanobodies) that bind to the cells comprising transmembrane proteins (e.g., GPCRs) may remain. In some embodiments, at least some of the complexes are amplified. In some embodiments, amplifying at least some of the complexes comprises amplifying the nucleic acids of the complexes. In some cases, amplifying at least some of the complexes comprises amplifying the cDNA of the complex.

A variety of methods for amplifying nucleic acids (e.g., DNA and/or mRNA) are suitable, in accordance with some embodiments. In some embodiments, amplifying the nucleic acids comprises bridge amplification. In some cases, amplifying the nucleic acids comprises polymerase chain reaction (PCR). For example, the nucleic acids may be purified and added to a medium comprising a polymerase (such as Taq polymerase) and DNA nucleotides (deoxyribonucleotides), wherein the nucleic acids are treated (e.g., repeated heated and cooling) to amplify the nucleic acid within the droplets. Suitable reagents for PCR or other amplification techniques, such as polymerases and/or deoxyribonucleotides, may be added to the solution initially, during, or after amplification. Therefore, in some embodiments, nucleic acids which encode for proteins that bind to cells comprising transmembrane proteins may be amplified. Other methods for amplifying nucleic acids are possible.

As noted above, the nucleic acids (e.g., mRNA and/or cDNA) that encode for proteins of interest (e.g., nanobodies) may be amplified and then used as a second gene library. That is, a second gene library may comprise nucleic acids (e.g., cDNA from a previous or first gene library), which can be transcribed to form more nucleic acids (e.g., a third nucleic acid, a fourth nucleic acid), which may encode for proteins that selectively target transmembrane proteins as described elsewhere herein. In some embodiments, it may be useful to repeat the process for the first gene library for the second gene library to further select genes which encode for proteins that bind to transmembrane proteins and/or modulate the activity of the transmembrane proteins. In other words, in some embodiments, a method further comprises transcribing the nucleic acids in the second gene library to synthesize a third nucleic acid (mRNA), binding another linking reagent (e.g., puromycin) to the third nucleic acid, translating the third nucleic acid to synthesize proteins (e.g., nanobodies), and/or further identifying proteins which bind to and/or modulate the activity of cells comprising transmembrane proteins of interest can be useful.

The number of genes that selectively bind to transmembrane proteins (e.g., GPCRs) that are selected from the second gene library may be less than the number of genes initially in the second gene library. In some cases, the number of genes selected from the second gene library is relatively large, being greater than or equal to 1, greater than or equal to 10¹, greater than or equal to 10², greater than or equal to 10³, greater than or equal to 10⁴, greater than or equal to 10⁵, greater than or equal to 10⁶, greater than or equal to 10⁷, greater than or equal to 10⁸, greater than or equal to 10⁹, greater than or equal to 10¹⁰, greater than or equal to 10¹¹, or greater than or equal to 10¹² complexes bound to cells. In some cases, the number of genes selected from the second gene library may be less than or equal to 10¹², less than or equal to 10¹¹, less than or equal to 10¹⁰, less than or equal to 10⁹, less than or equal to 10⁸, less than or equal to 10⁷, less than or equal to 10⁶, less than or equal to 10⁵, less than or equal to 10⁴, less than or equal to 10³, less than or equal to 10², less than or equal to 10¹, or less than or equal to 1 complex bound to the cells. Combinations of these ranges are possible (e.g., greater than or equal to 10² and less than or equal to 10⁵). Other ranges are also possible.

The genes selected from the second gene library may then again be used to form a third gene library, a fourth gene library, a fifth gene library, and so forth. In some embodiments, genes selected from subsequent libraries may be selected in a manner similar to selection from the first and/or second gene library. The method may be iterated continuously until the gene library encodes for proteins that bind to and/or modulate the activity of a substrate of interest (e.g., transmembrane proteins, GPCRs).

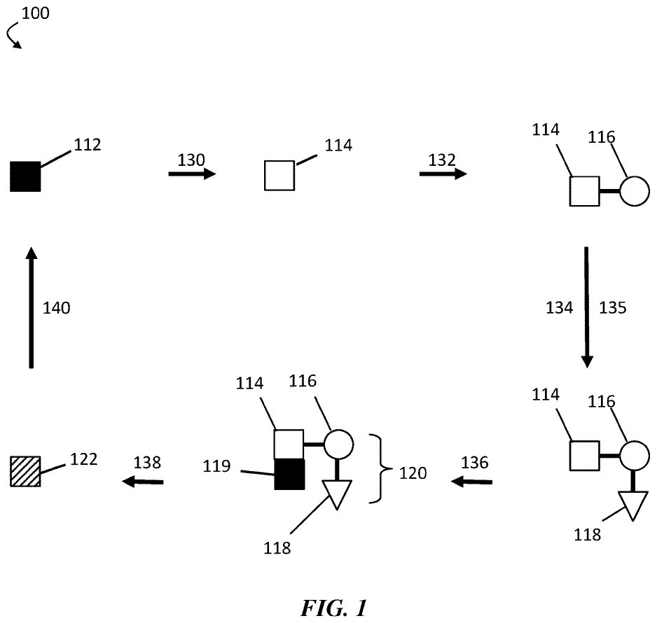

A non-limiting example of a method for screening (e.g., prescreening) a GPCR, or some other target, is schematically shown in FIG. 1. FIG. 1 shows a method 100 for screening a gene library. The gene library comprises genetic material 112, which is transcribed (i.e., transcription 130 in the figure) into a first nucleic acid 114, wherein the first nucleic acid is ligated (i.e., shown as bond 132 in the figure) to a linking reagent 116. The first nucleic acid 114 is then translated (shown as translation 134 in the figure) to synthesize a protein, such as a nanobody 118, for which it encodes, wherein the nanobody is transferred 135 from the first nucleic acid 114 to the linking reagent 116. The first nucleic acid 114 is then transcribed (e.g., via reverse transcription) to synthesize a second nucleic acid 119, complementary to the first nucleic acid 114, which base pairs with the first nucleic acid to form a complex 120 comprising the first nucleic acid 114, the second nucleic acid 119, the linking reagent 116, and the nanobody 118. The complex 120 is then sorted and washed (i.e., washing process 138 in the figure) to identify genetic material (e.g., the second nucleic acid 119 from complex 120) of interest 122 that encodes for the nanobody 118 that binds to GPCRs. The genetic material obtained following 138 may form another gene library to repeat the method 100 with this generated gene library and to further screen and identify genetic material that encodes for nanobodies that bind to GPCRs.

As discussed herein, there are a variety of suitable methods by which proteins (e.g., nanobodies) may bind to transmembrane proteins (e.g., GPCRs). Non-limiting examples of binding include hydrogen bonding, van der Waals forces, and/or forming covalent bonds (e.g., covalent and/or ionic bonds) between the proteins and transmembrane proteins.

Systems and methods are configured such that upon binding of one or more proteins (e.g., nanobodies) to a transmembrane protein (e.g., a GPCR), a signaling cascade mediated by the transmembrane protein is modulated (e.g., activated or deactivated), in accordance with some embodiments. In some cases, upon binding of one or more proteins to the transmembrane protein, the one or more proteins act as an agonist and activates the signaling cascade. In some cases, upon binding of one or more proteins to the transmembrane protein, the one or more proteins act as an antagonist by deactivating the signaling cascade. In some embodiments, binding of one or more nanobodies to a GPCR modulates a GPCR signaling cascade. In some such embodiments, the one or more nanobodies act as an agonist and/or an antagonist to the GPCR, activating and/or deactivating the GPCR signaling cascade.

A signaling cascade (e.g., a GPCR signaling cascade) that is mediated by a transmembrane protein (e.g., a GPCR) can induce a variety of responses, in accordance with some embodiments. For example, in some cases, the signaling cascade comprises releasing an enzyme. In some embodiments, the signaling cascade comprises modulating (e.g., opening, closing) an ion channel. In some cases, the signaling cascade comprises a conformational change in the transmembrane protein and/or an adjacent transmembrane protein. The signaling cascade may comprise a chemical reaction. In some such cases, the chemical reaction comprises the transmembrane protein reacting and/or the transmembrane protein reacting with a species in solution. In some embodiments, a GPCR signaling cascade may activate a G protein (e.g., the G protein to which the GPCR is coupled). Other responses induced by the signaling cascade are possible, as this disclosure is not so limiting.

Methods for identifying proteins (e.g., nanobodies) that selectively bind transmembrane proteins (e.g., GPCRs) and modulate (e.g., activate, deactivate) signaling cascades (e.g., GPCR signaling cascades) may comprise using a signaling entity. For example, in some embodiments, the transmembrane protein may comprise the signaling entity. The signaling entity can have any number of appropriate structures, in accordance with a variety of embodiments. For example, in some cases, the signaling entity may comprise a species configured for Förster resonance energy transfer (FRET). In some embodiments, the signaling entity may comprise a species configured for electron transfer (i.e., an oxidizing or reducing agent). In some cases, the signaling entity may comprise a chromophore and be configured to change its absorbance spectrum. In some embodiments, a GPCR comprises the signaling entity. In some cases, the GPCR comprises a signaling entity configured for FRET. Other embodiments of the signaling entity are also possible, as this disclosure is not so limited.

Activating and/or deactivating the signaling entity is possible in a variety of methods, in accordance with some embodiments. In some embodiments, the signaling entity is configured to generate a signal upon binding of the protein of interest (e.g., nanobody) to the substrate of interest (e.g., GPCR). In some cases, the signaling entity is configured to generate the signal upon binding of the protein of interest to the substrate of interest and a subsequent activation of a signaling cascade (e.g., a GPCR signaling cascade) that is mediated by the substrate of interest. For example, in some cases, a nanobody binds to a GPCR, inducing the signal from the signaling entity. In other cases, a nanobody binds to the GPCR and modulates (e.g., activates, deactivates) a GPCR signaling cascade that is mediated by the GPCR, wherein the signaling entity then produces the signal.

A signal from a signaling entity can manifest in a variety of embodiments. In some cases, the signal from the signaling entity maybe electrochemical in nature. For example, in some embodiments, the signaling entity may have a change in oxidation state. In some such cases, upon one or more proteins (e.g., nanobodies) binding to and/or modulating a transmembrane protein (e.g., a GPCR), electron transfer is initiated that changes the oxidation state of the signaling entity, thereby inducing an electrical signal that may be measured at an electrode.

In some cases, a signal from a signaling entity may be chemical in nature. In accordance with some embodiments, upon one or more proteins (e.g., nanobodies) binding and/or modulating the activity of a transmembrane protein (e.g., GPCR), the signaling entity reacts with another species in solution, thereby inducing a chemical change in the species in solution. In some such cases, chemical reactions that occur between the species in solution and the signaling entity may result in another detectable property (e.g., a signal). For example, in accordance with some embodiments, the species in solution that has reacted with the signaling entity may have a change in its absorbance spectrum. In some embodiments, the species in solution that has reacted with the signaling entity may begin to fluoresce. In some embodiments, the species in solution that has reacted with the signaling entity may have a change in its fluorescence. In some embodiments, a species in solution that has reacted with the signaling entity may precipitate from solution. Other chemical signals are possible, as this disclosure is not so limiting.

In some cases, a signal from a signaling entity may be optical. In some embodiments, the signaling entity may be a chromophore wherein, upon one or more proteins (e.g., nanobodies) binding to and/or modulating the activity of a transmembrane protein (e.g., GPCR), the absorbance of the chromophore changes. In some cases, the signal from the signaling entity may comprise fluorescence. In some embodiments, the signal from the signaling entity may comprise phosphorescence. In some cases, the signal from the signaling entity comprises shifting a wavelength of fluorescence and/or phosphorescence upon binding of one or more proteins to the transmembrane proteins. In other words, in accordance with some embodiments, the signaling entity may initially fluorescence, but upon binding of one or more proteins to the transmembrane protein, the signaling entity may fluoresce at a different wavelength than it did before the binding of the protein. In some cases, the signaling entity is configured for FRET, enabling a shift in fluorescence of the signaling entity upon binding of one or more proteins to the transmembrane protein. In some embodiments, a GPCR comprises the signaling entity comprising a molecule or moiety configured for FRET, wherein, upon binding of a nanobody to the GPCR, a GPCR signaling cascade is initiated and the molecule or moiety configured for FRET fluoresces and/or its fluorescence spectrum changes.

In some cases, binding of a protein of interest (e.g., a nanobody) to a substrate of interest (e.g., a transmembrane protein, a GPCR) can be detected by determining a signal from the signaling entity. Determining the signal from the signaling entity can be done by a variety of suitable approaches, in accordance with some embodiments. Optical signals from the signaling entity may be observed with a microscope, in some cases. In other cases, optical signals may be detected using a detector (e.g., a CCD or a CMOS camera, a photomultiplier tube). In some cases, detection is colorimetric, and a user may visually observe modulation of a GPCR by a color change (e.g., via a fluorescent signal generated after binding of protein to the GPCR). In some embodiments, optical signals (e.g., changes in absorbance spectra, a precipitate forming) may be detected visually. In some cases, changes in absorbance of the signaling entity and/or the solution may be measured using a UV-vis spectrometer. In some embodiments, electrochemical signals from the signaling entity may be measured at an electrode. Other detection schemes are also possible.

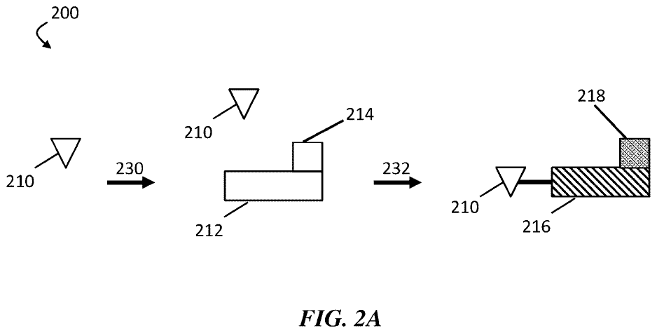

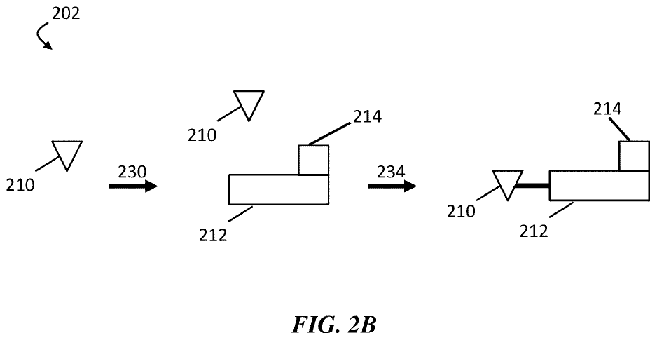

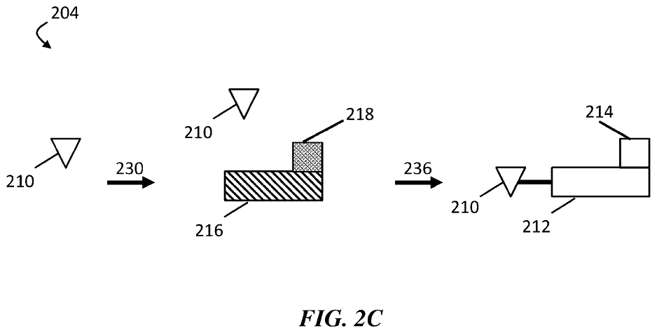

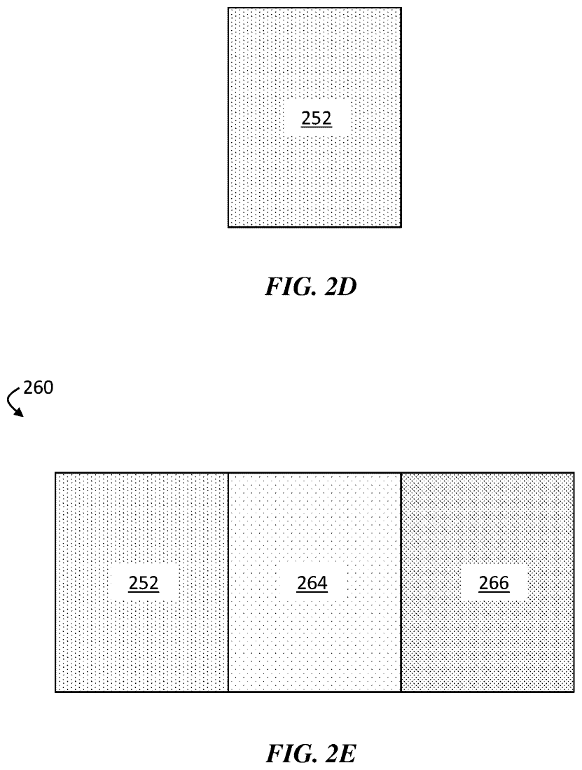

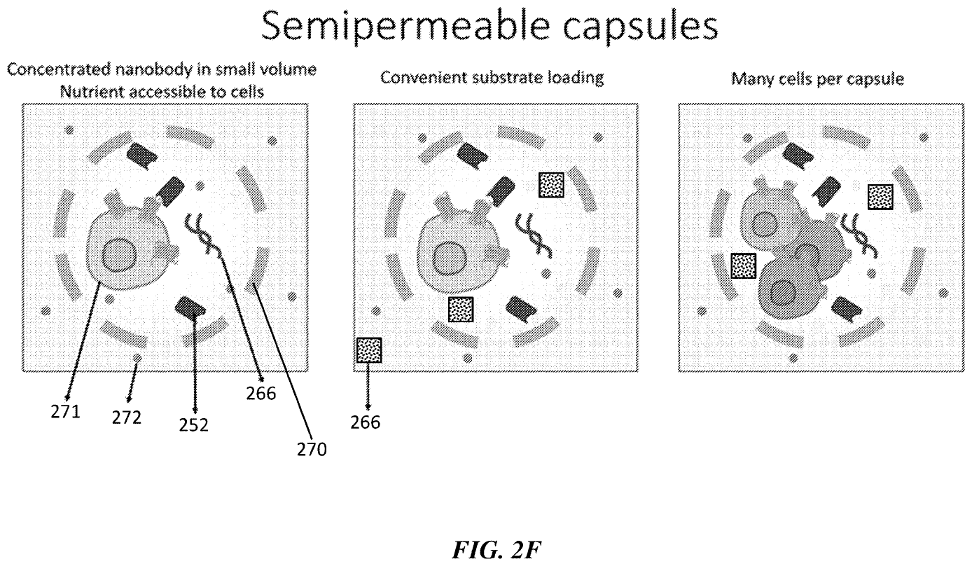

Turning now to FIGS. 2A-2E, non-limiting examples of proteins binding to and/or modulating the activity of transmembrane proteins are illustrated. FIG. 2A shows a method 200, wherein a nanobody 210 is exposed to an inactive GPCR of interest 212 coupled with a signaling entity comprising a moiety 214, which may interact with the signaling entity to generate a signal (e.g., a fluorescent signal). A process 230, such as flowing nanobody 210 may cause nanobody 210 to come into contact and/or bind to the GPCR 212. Upon the nanobody binding to and activating the GPCR (i.e., process 232), the GPCR of interest is activated 216 (e.g., activating a GPCR signaling cascade) and the signaling entity signals 218 (e.g., the signaling entity may fluoresce as a result, at least in part, of binding of the nanobody). In some such cases, the signal from the signaling entity is detected, which indicates that the nanobody 210 has bound to the GPCR and/or activated the GPCR.

FIGS. 2B and 2C show exemplary embodiments that are similar to the embodiment shown in FIG. 2A. In FIG. 2B, method 202 shows that, upon binding 234 of the nanobody 210 to the inactive GPCR of interest 212, the GPCR remains inactive (e.g., no GPCR signaling cascade is activated) and the signaling entity does not signal. In contrast, in FIG. 2C, a method 204 shows the nanobody 210 being exposed to an active GPCR 216 (e.g., a GPCR activating a GPCR signaling cascade) and a signaling entity producing a signal 218. Upon binding of the nanobody to the active GPCR and incubating 236, the GPCR is deactivated 212 (e.g., the GPCR signaling cascade is deactivated) and the signal from the signaling entity stops (e.g., fluorescence stops).

FIG. 2D is a non-limiting example schematically showing a nanobody 252 suspected of binding a GPCR and modulating the activity of a GPCR signaling cascade. In some embodiments, described herein and illustrated as a non-limiting example in FIG. 2E, a complex 260 is formed comprising the nanobody 252, a linking reagent 264, and a strand of a nucleic acid 266, wherein the strand of nucleic acids in the complex encodes for the nanobody in the complex. By using the complex illustrated in FIG. 2E, screening and identifying nanobodies capable of binding and/or modulating the activity of GPCRs of interest also may provide the genes encoding for the screened nanobodies.

In some embodiments, one or more nanobodies and/or cells reporting one or more nanobodies of interest are at least partially (e.g., completely) encapsulated by a semipermeable membrane. The semipermeable membrane permits entry of some species while rejecting entry of some other species. In some such embodiments, the semipermeable membrane provides a microenvironment for cell growth (for example, in live-cell reporter assay) by allowing nutrients to diffuse toward cells within the semipermeable membrane (which may also include or encapsulate a nanobody of interest) while blocking larger molecules from entering or exiting that are not necessarily required for cell growth. For example, in FIG. 2F, nanobody of interest 252 is within a semipermeable membrane 270, along with a GPCR reporter cell 271. Nutrients 272 can diffuse in to and/or out of the semipermeable membrane 270, but larger molecules, such as the nanobody 252 and/or the nucleic acid 266 cannot. This may advantageously improve cell health of a reporter cell, facilitate the addition of small-molecule reagents to the cells while still maintaining a relatively high microenvironment of nanobodies around the cell, and/or provide for multi-cell encapsulation so that several or more cells can be screened simultaneously (or essentially simultaneously). Techniques for encapsulating nanobodies or cells, etc. in a semipermeable membrane, e.g., in a single or double emulsion droplet, will be known by those of ordinary skill in the art. See, for example, U.S. Pat. No. 10,316,873 or 11,141,731, each incorporated herein by reference in its entirety.

In some embodiments, a fluidic system may be used to perform some or all of the method steps described above. “Microfluidic,” as used herein, refers to a device, apparatus, or system including at least one fluid channel having a cross-sectional dimension (measured perpendicular to the direction of fluid flow) of less than 1 millimeter (mm), and in some cases, the ratio of length to the largest cross-sectional dimension of at least 3:1.

A “channel,” as used herein, refers to a feature on or in a system that at least partially directs flow of a fluid. The channel can have any cross-sectional shape (e.g., circular, oval, triangular, square, rectangular, irregular, etc.) and can be covered or uncovered. One or more channels may (but not necessarily), in cross section, have a height that is substantially the same as a width at the same point.

In embodiments where it is completely covered, at least one portion of the channel can have a cross section that is completely enclosed, or the entire channel may be completely enclosed along its entire length with the exception of its inlet(s) and/or outlet(s). A channel may also have an aspect ratio (length to average cross-sectional dimension of at least 2:1, more typically at least 3:1, 5:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than 5 mm or 2 mm, or less than 1 mm, or less than 500 microns, less than 200 microns, less than 100 microns, less than 60 microns, less than 50 microns, less than 40 microns, less than 30 microns, less than 25 microns, less than 10 microns, less than 3 microns, less than 1 micron, less than 300 nm, less than 100 nm, less than 30 nm, or less than 10 nm. In some cases, the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

In some embodiments, fluidic droplets are formed by flowing 2, 3, or more fluids through a system of channels of a fluidic system. Some embodiments comprise a droplet contained within a carrying fluid. For example, there may be a first phase forming droplets contained within a second phase. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

The fluidic droplets within the channels may have a cross-sectional dimension smaller than 100% of an average cross-sectional dimension of the channel, and in certain embodiments, smaller than 90%, smaller than 80%, 70%, 60%, 50%, 40%, 30%, 20%, 10%, 5%, 3%, 1%, 0.5%, 0.3%, 0.1%, 0.05%, 0.03%, or 0.01% of the average cross-sectional dimension of the channel.

During use, at least in some embodiments, droplets may be used in a microfluidic system. Microfluidic systems may comprise a variety of materials, and those skilled in the art are able to select materials appropriate for forming the microfluidic system. For example, in some cases, the microfluidic system comprises glass. In other embodiments, the microfluidic system comprises hydrogels. In other embodiments, the microfluidic system comprises paper. In other embodiments, the microfluidic system comprises polymers. Non-limiting examples of polymers suitable for microfluidic systems include polydimethylsiloxane (PDMS) and polytetrafluoroethylene (PTFE). In some embodiments, the microfluidic device comprises metals. A non-limiting example of a metal suitable for microfluidic systems is stainless steel. Microfluidic devices comprising multiple materials (e.g., a hybrid glass-PDMS device) are possible. Using other materials is also possible.

Moreover, within a microfluidic system, droplets may be fluidically connected to one or more reservoirs of the fluidic system (e.g., to a pool used to form droplets, to a hydrophobic fluid used to form droplets, to a supply of a target substrate, to a supply of a detection agent, to a supply of in vitro transcription and translation reagents, or any of a variety of other fluids described herein). For example, the droplets may be connected to one or more reservoirs of a fluidic system via the microchannel. In some embodiments, the fluidic system comprises one or more additional components, such as a pressure source (for example, a pump), a detection tool (e.g., a sensor that may be used to detect fluorescence, luminescence, and/or colorimetric changes resulting from activity of a target substrate), and/or a waste stream.

In some embodiments, a droplet microfluidic platform is used to screen a gene library for genes encoding proteins that bind to transmembrane proteins. In some cases, screening the gene library comprises encapsulating the nucleic acids within a first plurality of microfluidic droplets. In some embodiments, individual droplets may encapsulate any number of genes (e.g., a nucleic acid, DNA, RNA) encoding for different proteins (e.g., mRNAs encoding for nanobodies). For example, an individual droplet may encapsulate greater than or equal to 1,000, greater than or equal to 10,000, greater than or equal to 100,000, greater than or equal to 1,000,000 genes. Alternatively, an individual droplet may encapsulate less than or equal to 1,000,000, less than or equal to 100,000, less than or equal to 10,000, or less than or equal to 1,000 genes. Combinations of these ranges are possible (e.g., greater than or equal to 1000 and less than or equal to 100,000 genes encapsulated in an individual droplet). Other ranges are also possible.

In some embodiments, the microfluidic droplets further comprise an in vitro transcription and translation mixture, cells, and a substrate of interest (e.g., a transmembrane protein, a GPCR). In some cases, the transcription and translation mixture comprises RNA polymerase, transcription factors, DNA comprising promoter DNA, RNA nucleotides, tRNA, ribosomes, and/or amino acids. In some embodiments, the cells comprise mammalian-derived cells. In some cases, the substrate of interest comprises a transmembrane protein. In some cases, the cells comprise the substrate of interest. In some embodiments, mammalian-derived cells comprise transmembrane proteins comprising GPCRs. In some such embodiments, the GPCRs are human derived. In this manner, in some embodiments, nucleic acids contained within a first plurality of microfluidic droplets can be incubated to translate and/or transcribe the nucleic acids to synthesize the proteins for which they encode. In some embodiments, the first plurality of microfluidic droplets contains proteins of interest (e.g., proteins suspected of binding GPCRs). In some cases, it is useful to encapsulate one or more proteins within a microfluidic droplet, wherein at least one of the proteins comprises a promoter and is suspected of being capable of binding to the substrate of interest.

In some embodiments, it may be useful to expose proteins contained within a first plurality of microfluidic droplets to a transmembrane protein (e.g., a GPCR) coupled to a signaling entity, whereby at least one of the proteins within the microfluidic droplet binds to the transmembrane protein coupled to the signaling entity. In some such cases, binding of the at least one protein to the transmembrane protein modulates a signaling cascade (e.g., a GPCR signaling cascade), wherein modulating the signaling cascade induces a signal (e.g., fluorescence) from the signaling entity. In some embodiments, DNA is transcribed to mRNA, which is in turn translated to the nanobodies for which it encodes within a microfluidic droplet. In some such cases, at least one of the nanobodies binds to a GPCR and/or modulates a GPCR signaling cascade that is mediated by the GPCR, thereby inducing the signal from the signaling entity. As mentioned elsewhere herein, the signal from the signaling entity may be present in a variety of embodiments, and the signal may be observed or measured in a number of suitable approaches, in accordance with various embodiments.

In some cases, wherein mammalian cells comprising a transmembrane protein (e.g., a GPCR) are used, detecting a signal that from a signaling entity may be advantageous for determining the activity of transmembrane proteins in their native configuration within their native environment. That is, the signal from the signaling entity indicates that a protein of interest (e.g., a nanobody) binds to and/or modulates the activity of the transmembrane protein, which can provide information about the activity of the transmembrane protein within its native environment (e.g., within a cell, within a cell membrane), in accordance with some embodiments.

In some cases, when using microfluidic droplets, after determining a signal from a signaling entity that indicates a protein of interest (e.g., a nanobody) is binding to the substrate of interest (e.g., a transmembrane protein, a GPCR) and/or modulating a signaling cascade (e.g., a GPCR signaling cascade) within a droplet of a first plurality of droplets, such droplets may be sorted from the first plurality of droplets to form a second plurality of droplets. In some embodiments, a nanobody binds to and/or modulates the activity of a GPCR, thereby activating a signaling entity configured for FRET. In some such embodiments, the signaling entity initially fluoresces with a green wavelength of light and, upon the nanobody binding to and/or modulating the activity of the GPCR, the signaling entity fluoresces blue light. In this manner, the ratio of green-to-blue light fluorescing from the droplet may indicate the droplet contains at least one nanobody that binds to and/or modulates the activity of the GPCR. Accordingly, in some such cases, droplets are sorted by the ratio of green-to-blue light fluorescing from the droplets. In some cases, droplets with a relatively high green-to-blue light ratio (e.g., green:blue is 1:0) are selected. In other cases, droplets with a relatively low green-to-blue light ratio (e.g., green:blue is 0:1) are selected.

Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in magnitude, direction, frequency, duration, etc.

By sorting a first plurality of droplets, in some embodiments, a second plurality of droplets may contain a number of different genes which encode for proteins of interest (e.g., nanobodies that bind to and/or modulate the activity of GPCRs). In some cases, the second plurality of droplets may contain a relatively large number of genes, including greater than or equal to 1, greater than or equal to 5, greater than or equal to 10, greater than or equal to 50, greater than or equal to 100, and greater than or equal to 1000 genes. In some embodiments, the second plurality of droplets may contain less than or equal to 1000, less than or equal to 100, less than or equal to 50, less than or equal to 10, less than or equal to 5, or less than or equal to 1 gene. Combinations of the aforementioned ranges are possible (e.g., greater than or equal to 10 and less than or equal to 50 genes). Other ranges are possible.

After sorting the first plurality of droplets to yield the second plurality of droplets, the second plurality of droplets may be combined, in accordance with some embodiments. By purifying the contents of the second plurality of droplets, in some embodiments, the genes which encode for the proteins of interest (e.g., a nanobody) that bind to the substrates of interest (e.g., a transmembrane protein, a GPCR) may be obtained. In some cases, the genes that are obtained by purifying the contents of the second plurality of droplets may be encapsulated into an additional plurality of droplets (e.g., a third plurality of droplets, a fourth plurality of droplets), whereby the contents of the additional plurality of droplets can be once again translated and transcribed to form proteins, which can be screened further. In some embodiments, the method for selecting proteins of interest may be repeated until the number of remaining genes is 1. In other words, in some embodiments, the methods disclosed elsewhere herein may be used to screen a gene library for the gene (or genes) which encodes for the protein (or proteins) that binds to and activates the substrate of interest.

In some embodiments, a first plurality of genes encoding for proteins that bind to and/or modulate the activity of a first plurality of GPCRs is selected from a gene library. In some cases, the foregoing method steps may be repeated to screen the proteins encoded within the first plurality of genes for cross reactivity to a second plurality of GPCRs. That is, in some cases, the first plurality of genes is translated and transcribed to obtain the proteins for which it encodes, which are then exposed to a second plurality of GPCRs which is different than the first plurality of GPCRs. In some such cases, the proteins of interest may be encapsulated in microfluidic droplets with the second plurality of GPCRs. In some such cases, GPCRs of the second plurality of GPCRs comprise a signaling entity, which produces a signal that indicates whether the proteins bind to and/or modulate the activity of the second plurality of GPCRs. In some cases, proteins that bind to and/or modulate the activity of the second plurality of GPCRs are selected. In some embodiments, proteins that do not bind to and/or modulate the activity of the second plurality of GPCRs are selected. In some cases, proteins that bind to and/or modulate the activity of the second plurality of GPCRs are not selected. In some cases, proteins that do not bind to and/or modulate the activity of the second plurality of GPCRs are not selected. In some cases, identifying GPCRs that bind and/or modulate GPCRs of the first plurality of GPCRs but not GPCRs of the second plurality of GPCRs may be advantageous for selecting proteins that do not cross react. Further screening the proteins with a third plurality of GPCRs, a fourth plurality of GPCRs, a fifth plurality of GPCRs, and so forth is possible and may help further select for proteins that bind to and/or modulate the activity of GPCRs of the first plurality of GPCRs but do not cross react with other GPCRs.

In some embodiments, the methods described herein can be performed sequentially and/or in tandem to screen gene libraries for genes (e.g., DNA, RNA, mRNA) encoding for proteins (e.g., nanobodies) that bind and/or modulate the activity of transmembrane proteins (e.g., GPCRs). For example, in some cases, a gene library may be screened as illustrated in FIG. E1, optionally repeating the cyclical screening process to select genes that encode for nanobodies that bind GPCRs. In some cases, the methodology for screening using microfluidic droplets can be used. In some cases, a relatively large gene library (e.g., comprising up to 10¹⁵ genes) can be screened as in FIG. E1 until there are fewer genes remaining (e.g., 1,000,000 genes) than in the initial gene library. In some such cases, the remaining genes are further screened using the microfluidic droplet approach leveraging signals from signaling entities described elsewhere herein until there are a relatively small number of genes selected. Non-limiting examples of the number of genes that may remain after screening are described above. In some cases, at most 10,000, at most 1,000, at most 100, at most 10, at most 5, or at most 1 gene remain after screening using the droplet methodology. Thus, using the methods disclosed herein, it is possible to begin with a relatively large gene library (e.g., 10¹⁵ genes) and select a relatively small number of genes (e.g., 10 genes) that encode for proteins that bind to and/or modulate the activity of transmembrane proteins of interest. Additionally, other methodologies are possible.

In some embodiments, droplets containing a protein and/or a nucleic acid of interest (e.g., a first nucleic acid, a second nucleic acid) may be pooled. For example, the droplets can be pooled (e.g., combined, merged, coalesced) using methods known in the art. In some embodiments, the pooled droplets (e.g., proteins and/or nucleic acids within the droplets) can be combined to form a second library (e.g., a second gene library). In some such embodiments, the second library can be screened to form a third library (e.g., a third gene library), wherein the amount of proteins and/or nucleic acids of interest within the third library is less than the amount of proteins and/or nucleic acids of interest within the second library.

As mentioned above, the systems and methods described herein are suitable for screening proteins (e.g., nanobody) that bind and/or modulate the activity of a transmembrane protein (e.g., GPCR). However, it should be understood that the systems and methods can also be used to screen the activity of other binding agents and their related activity to a receptor. For example, in some embodiments, other transmembrane proteins are screened, such transmembrane transporters, receptors, enzymes, and anchors. Non-limiting examples include pore-forming membranes proteins, such as ion channel proteins (e.g., proton channels, sodium channels, calcium channels, potassium channels, other cation channels, voltage-gated ion channels, transient receptor channels) and enzyme-linked receptors (e.g., ligand-gated ion channels, ionotropic receptors, acetylcholine receptors, ionotropic glutamate-gated receptors, acid sensing ion channels (ASICs), ATP-gated P2X receptors, GABAA receptors). Additional non-limiting transmembrane proteins include transmembrane proteins that permit the transport of specific substances across the membrane such as light absorption-driven transporters (e.g., rhodopsin), redox-driven transporters (e.g., transmembrane cytochrome b-like proteins such as coenzyme Q), electrochemically driven transporters (e.g., ATPases), bond-forming or bond-breaking transporters (e.g., phospholamban), porters (e.g., mitochondrial carrier proteins, neurotransmitter sodium symporter, symport/antiport proteins such as glucose transporters). Other transmembrane proteins are possible and, as noted elsewhere herein, non-transmembrane proteins are also possible, as this disclosure is not so limited.

U.S. Provisional Patent Application Ser. No. 63/432,606, filed Dec. 14, 2022, and U.S. Provisional Patent Application Ser. No. 63/432,600, filed Dec. 14, 2022 are incorporated herein by reference in their entireties.

The following examples are intended to illustrate certain embodiments of the present invention, but do not exemplify the full scope of the invention.

### Example 1

The following example describes a system for screening for nanobodies that bind to and modulate the activity of GPCRs.

GPCRs are categorized into six classes based on sequence and function, namely Class A—rhodopsin-like receptors, Class B—secretin family, Class C—metabotropic glutamate receptors, Class D—fungal mating pheromone receptors, Class E—cAMP receptors, and Class F—frizzled (FZD) and smoothened (SMO) receptors. In the case of class A GPCRs, the endogenous ligand is recognized by a ligand-binding site in the 7TM region. For class B GPCRs, the ligand is recognized by both extracellular and 7TM domains. For class C GPCRs, the ligand-binding pocket is found in the extracellular domain (ECD) that contains a Venus flytrap (VFT) module. In the case of class F GPCRs, both SMO and FZD receptors possess an ECD that is comprised of an extracellular cysteine-rich domain (CRD) and an ECD linker domain. The endogenous lipoglycoprotein ligand, Wnt, binds to the CRD of the FZD receptors. Upon ligand binding, GPCRs activate at least one of the two signaling partners, namely heterotrimeric GTP-binding proteins (G-proteins) or β-arrestins, and mediate signal flow via modulation of various downstream effectors.

In 2019, there were 481 drugs (~34% of all drugs approved by the FDA) that acted at 107 unique GPCR targets. Approximately 320 agents are currently in clinical trials, of which ~36% target 64 potentially novel GPCR targets without an approved drug, and the number of biological drugs, allosteric modulators and biased agonists has grown. The major disease indications for GPCR modulators show a shift towards diabetes, obesity, and Alzheimer's disease, while other central nervous system disorders remain highly represented. The 227 (57%) non-olfactory GPCRs that are yet to be explored in clinical trials have broad untapped therapeutic potential, particularly in genetic and immune system disorders. Example drugs that act at GPCR targets are listed in Table 1.

TABLE 1
Some drugs approved by the FDA since 2014.

| Drug | Substance | Indication(s) | Target(s) | Approval |
|---|---|---|---|---|
| Hycodan | Hydrocodone | Narcotic cough | OPRD, OPRM | 2014 |
| | bitartrate | | | |
| Rexulti | Brexpiprazole | Depression | 5HT2A, 5HT1A, | 2015 |
| | | | DRD2, 5HT7R | |
| Trulicity | Dulaglutide | Diabetes, Type 2 | GLP1R | 2014 |
| Varubi | Rolapitant | Nausea/vomiting | NK1R | 2015 |
| Akynzeo | Netupitant | Nausea/vomiting | NK1R | 2014 |
| Uptravi | Selexipag | Pulmonary hypertension | PI2R | 2015 |
| Odomzo | Sonidegib | Basal cell carcinoma | SMO | 2015 |
| Tymlos | Abaloparatide | Osteoporosis | PTHR1 | 2017 |
| Nuplazid | Pimavanserin | Parkinson's disease | 5HT2A | 2016 |
| | | psychosis | | |
| Kengreal | Cangrelor | Percutaneous coronary | P2Y12 | 2015 |
| | | intervention | | |
| Zontivity | Vorapaxar | Cardiovascular risk | PAR1, PAR3, | 2014 |
| | | reduction | PAR4 | |
| Striverdi | Olodaterol | Chronic obstructive | ADRB2 | 2014 |
| respimat | | pulmonary disease (COPD) | | |
| Natpara | Parathyroid | Hypoparathyroidism | PTH1R | 2015 |
| | horomone | | | |
| Adlyxin | Lixisenatide | Diabetes, Type 2 | GLP1R | 2016 |
| Belsomra | Suvorexant | Insomnia | OX2R, OX1R | 2014 |
| Hetlioz | Tasimelteon | Non-24-hour disorder | MTR1A, MTR1B | 2014 |
| Viberzi | Eluxadoline | Irritable bowel syndrome | OPRM, OPRD | 2015 |
| Movantik | Naloxegol | Constipation | OPRM | 2014 |
| Symproic | Naldemedine | Constipation | OPRM | 2017 |
| Northera | Droxidopa | Orthostatic hypotension | ADRB1-3, | 2014 |
| | | | ADA2A/B/C, | |
| | | | ADA1A/B/D | |
| Parsabiv | Etelcalcetide | Hyperparathyroidism | CASR | 2017 |
| Aristada | Aripiprazole | Schizophrenia | 5HT1A, 5HT2A, | 2015 |
| | lauroxil | | DRD2 | |
| Vraylar | Cariprazine | Schizophrenia | DRD3, DRD2 | 2015 |
| Addyi | Flibanserin | Hypoactive sexual | 5HT2A, 5HT1A | 2015 |
| | | desire disorder | | |

### Methodology

A methodology to screen for proteins and nanobody therapeutics (e.g., agonists or antagonists of GPCRs) was developed based on a combination of conventional cellular assays, in vitro transcription and translation, and microfluidics. DNA libraries encoding for nanobodies were used with T7 promotor and/or terminators allowing for cell-free protein/protein expression.

A cellular assay based on mammalian cells expressing human GPCRs was used. Binding of a nanobody to the GPCR resulted in the activation of a signaling cascade, wherein beta-lactamase was expressed, leading to the cleavage of a FRET-based substrate and, in turn, a change of fluorescence of the FRET-based substrate from green to blue. In other words, in the presence of the nanobody that bound and activated the GPCR, the observed fluorescence from the FRET-based substrate shifted from green to blue. This allowed microfluidic sorting of droplets containing proteins that did or did not bind to the GPCRs of interest based on an observed green-blue fluorescence ratio.

Up to 1 million genes encoding for different proteins were encapsulated into individual droplets together with an in vitro transcription and translation mixture, cells, and substrate. The droplets were incubated, which allowed transcribed nanobodies to bind and activate GPCR substrates, express beta-lactamase, and cleave a FRET-based substrate bound to the GPCR. After which, droplets with high blue-to-green (indicating nanobodies binding and activating GPCRs) fluorescence ratios were sorted. Sorted droplets were merged and the DNA within the droplets was multiplied, providing about 10000 copies of sorted genes. The 10000 copies of sorted genes were then encapsulated into droplets and the incubation/sorting process was repeated, providing about 100 copies of sorted genes. Again, the genes were encapsulated, incubated, and sorted leaving only 1 gene in every tenth droplet. Genes from this final sorting process were taken for further testing and/or for application as therapeutics.

### Example 2

The following example describes a system for pre-screening a gene library comprising up to 10¹⁵ DNA species for nanobodies that bind to and/or modulate the activity of GPCRs.

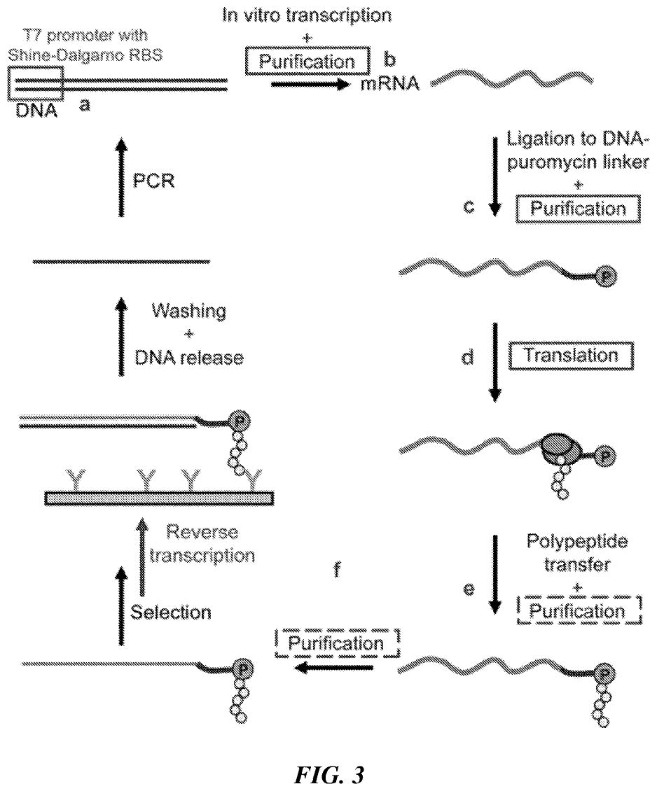

A methodology was developed for pre-screening a gene library comprising DNA molecules, wherein the DNA molecules encoded for nanobodies which were suspected to bind and activate GPCRs (e.g., cells expressing target GPCRs). FIG. 3 shows a schematic diagram illustrating this method. The library comprised up to 10¹⁵ distinct DNA molecules. The DNA was transcribed to mRNA, which was then purified. FIG. 3 shows a schematic diagram illustrating this method. Subsequent ligation of the mRNA to a DNA-puromycin linker was performed, whereafter the mRNA-DNA-puromycin was purified. The mRNA in the mRNA-DNA-puromycin was then translated to form a nanobody, which was transferred onto the puromycin forming a first plurality of complexes comprising mRNA and the nanobody for which the mRNA encoded. The mRNA of the first plurality of complexes was then reverse transcribed to form cDNA, which base paired with the mRNA to form a second plurality of complexes. The second plurality of complexes was exposed to cells expressing GPCRs of interest, wherein the cells were immobilized on a substrate. The cells were then washed after exposure to the second plurality of complexes, which released the cDNA from the second plurality of complexes which encoded for nanobodies that targeted the GPCRs of interest. The cDNA was then amplified with polymerase chain reaction (PCR). The foregoing process was then repeated to further select the nanobodies that bind and activate target GPCRs which were expressed in the immobilized cells.

### Example 3

The following example describes the usage of semipermeable microcapsules as screening vehicles for reporting GPCR activity when the appropriate nanobody binds to the GPCR.

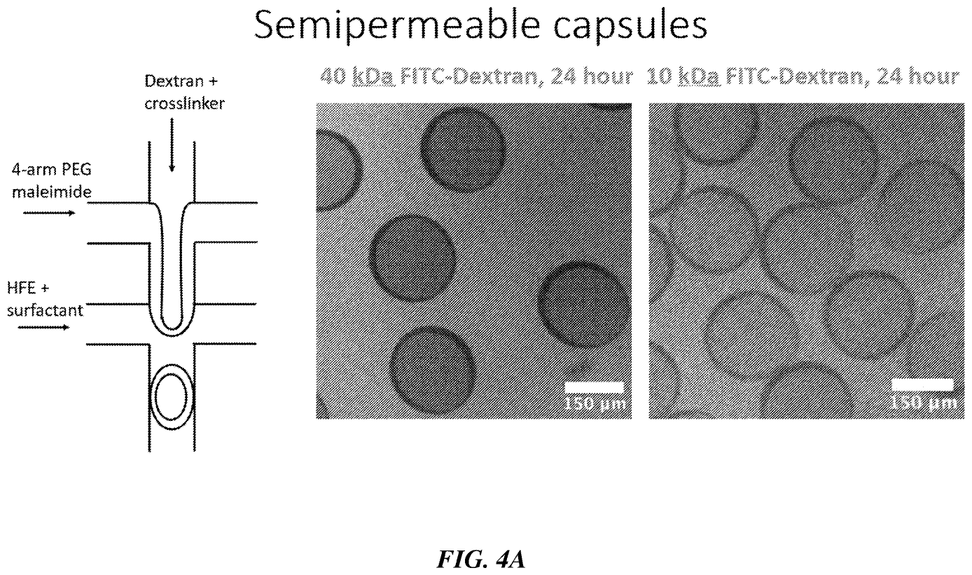

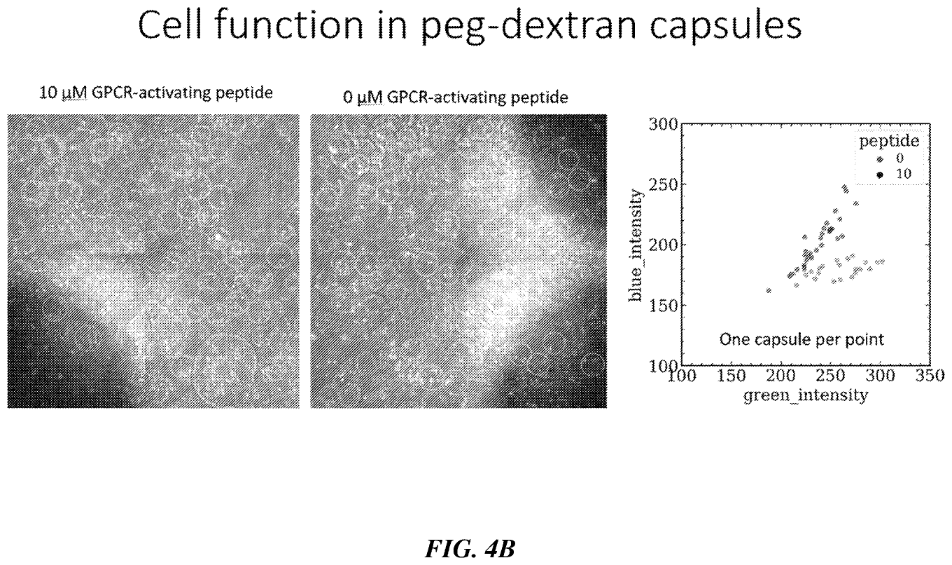

A live-cell reporter assay was developed to provide the cells reporting GPCR activity upon binding had sufficient nutrients. Cells reporting the activity of the nanobody were encapsulated in a semipermeable dextran membrane, as shown in FIG. 4A. During the assay, nutrients could move towards or away from the cell within the semipermeable microcapsule by diffusing through the semipermeable membrane while larger molecules (e.g., the nanobodies and the genes that encode for them) could not diffuse through the semipermeable membrane. The semipermeable microcapsule, therefore, allowed small molecules to exchange in to or out of the membrane but kept the peptides and the genes inside the capsule, closer to the reporter cell.

The cell reporter turns “on” when the appropriate nanobody is provided. For example, as shown in FIG. 4B, the fluorescence intensity increases when the GPCR-activating peptide (a nanobody) is provided is “off” relative to when no GPCR-activating peptide is present.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for determining proteins that bind to GPCRs, the method comprising:
containing one or more proteins within a microfluidic droplet, wherein at least one of the proteins is suspected of being capable of binding to a signaling entity;
binding at least one of the proteins contained within the microfluidic droplet to a GPCR receptor coupled to the signaling entity, wherein the binding activates the signaling entity; and
determining a signal from the signaling entity.

2. The method of claim 1, wherein binding activates the signaling entity.

3. The method of any one of claims 1-2, further comprising initiating a GPCR cascade that activates the signaling entity.

4. The method of any one of claims 1-3, further comprising initiating a GPCR cascade that activates the signaling entity after binding of one or more proteins to the GPCR receptor.

5. A method for determining proteins that bind to GPCRs, the method comprising:
containing one or more proteins within a microfluidic droplet, wherein at least one of the proteins is suspected of being capable of binding to a signaling entity;
binding at least one of the proteins contained within the microfluidic droplet to a GPCR receptor coupled to the signaling entity; and
determining a signal from the signaling entity.

6. The method of claim 5, wherein binding deactivates the signaling entity.

7. The method of any one of claims 5-6, further comprising initiating a GPC cascade that deactivates the signaling entity.

8. The method of any one of claims 5-7, further comprising initiating a GPC cascade that deactivates the signaling entity after binding of one or more proteins to the GPCR receptor.

9. The method of any one of claims 1-8, wherein the one or more proteins comprises a nanobody.

10. The method of any one of claims 1-9, wherein the GPCR comprises a human-derived GPCR.

11. The method of any one of claims 1-10, wherein the signaling entity is configured to generate a signal upon binding of the proteins.

12. The method of any one of claims 1-11, wherein the signaling entity further comprises a species configured for Forster resonance energy transfer (FRET).

13. The method of any one of claims 1-12, further comprising containing the microfluidic droplet within a semipermeable membrane.
